(19) United States
(12) Patent Application Publication
(10) Pub. No.: US 2019/0225590 A1
(43) Pub. Date: Jul. 25, 2019

(54) METHOD FOR PREPARING FLUOROALKYLNITRILES AND THE CORRESPONDING FLUOROALKYLTETRAZOLES

(71) Applicant: BAYER CROPSCIENCE AKTIENGSELLSCHAFT, Monheim am Rhein (DE)

(72) Inventors: Claus-Christian HAESELHOFF, Gladbeck (DE); Albert SCHNATTERER, Leverkusen (DE); Jan VERMEHREN, Idstein (DE)

(73) Assignee: Bayer CropScience Aktiengesellschaft, Monheim am Rhein (DE)

(21) Appl. No.: 16/318,068

(22) PCT Filed: Jul. 20, 2017
(86) PCT No.: PCT/EP2017/068328

§ 371 Date: Jan. 15, 2019

(30) Foreign Application Priority Data
Jul. 28, 2016 (EP) 16181723.4

(51) Int. Cl. C07D 257/04

(52) U.S. Cl. CPC C07D 257/04; C07C 255/10

(57) ABSTRACT

The present invention relates to a method for preparing fluoroalkylnitriles and the corresponding fluoroalkyltetrazoles by reacting fluorinated carboxamides with phosphorus trichloride (PCl₃) or phosphorus oxychloride (POCl₃) in the presence of a base.

The present invention relates to a method for preparing fluoroalkylnitriles and the corresponding fluoroalkyltetrazoles starting from fluoroalkylcarboxamides.

Fluoroalkylnitriles and the corresponding fluoroalkyltetrazoles are important intermediates for preparing agrochemical active ingredients.

Grunewald et al. (J. Med. Chem. 2006, 49, 2939-2952) and also Swarts (Bulletin des Sociétés Chimiques Belges, 1922, Vol 31, 364-365) describe the preparation of difluoroacetonitrile starting from difluoroacetamide and phosphorus pentoxide. In this case, both solids are heated and the volatile nitrile is condensed at −78° C. The solid reaction residue that remains in the reaction vessel is, however, difficult to remove.

Parker in Synthetic Communications (Volume 34, 2004, pages 903-907) and EP 729940A2 describe the preparation of fluorinated nitriles by reacting the corresponding amides with trifluoroacetic anhydride in pyridine. A disadvantage of this process is the use of expensive trifluoroacetic anhydride which has to be used stoichiometrically.

CN 102746190A (2012) describes the preparation of trifluoroacetonitrile from the amide by the polyphosphoric acid/phosphoric acid catalyst system.

CN 103804231A (2014) discloses the preparation of trifluoroacetonitrile from the amide by addition of trifluoroacetic anhydride in carbon tetrachloride.

WO 2010/142377A1 describes the preparation of fluoroalkylnitriles by reacting fluorinated carboxamides with acid halides and fluorinated carboxylic acids.

A problem in using acid halides as dehydrating agents is that by-products such as hydrogen halide are formed. Since fluoroalkylnitriles are frequently reacted with, for example, sodium azide and acetonitrile to give the corresponding fluoroalkyltetrazoles (see also Radies in Journal of Fluorine Chemistry (2008, 129, 1199-1205)), the formation of the unstable and extremely explosive hydrogen azide (HN₃) by reaction of the corresponding hydrogen halide with the sodium azide is possible but actually absolutely undesirable.

Jones in Journal of Organic Chemistry (1943, 65, 1458) describes the dehydration of trifluoroacetamide by phosphorus pentoxide. All of the methods described above are characterized in that special apparatus, very high temperatures and expensive and hazardous reagents are required; the desired products can only be isolated by complicated isolation from the product mixtures.

DE 69428783T2 discloses, in addition to other dehydrating agents, also phosphorus oxychloride (POCl₃) as suitable for preparing carbonitriles. Fluorinated derivatives are however not described therein.

Proceeding from this prior art, the object of the present invention is to provide a safest possible method (i.e. completely avoiding the formation of HN₃ for example) for preparing fluorinated alkylnitriles and the fluorinated alkyltetrazoles obtainable therefrom, which can be carried out preferably in a simple and cost-effective manner. The fluorinated alkylnitriles and fluorinated alkyltetrazoles obtainable by this desired method should preferably in this case be obtained in high yield and high purity. In particular, the desired method should enable the desired target compounds to be obtained without the need for complex purification methods.

The object according to the present invention was achieved by a method for preparing fluoroalkylnitriles of the general formula (I),

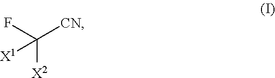

in which

X¹ and X² are mutually independently fluorine, chlorine, hydrogen or methyl,

characterized in that

fluorinated carboxamides of the formula (II)

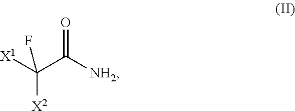

in which X¹ and X² are as defined above,

are reacted with phosphorus trichloride (PCl₃) and/or phosphorus oxychloride (POCl₃) in the presence of a base.

X¹ and X² are each independently preferably fluorine.

In a preferred embodiment of the invention, the fluoroalkylnitriles of the general formula (I),

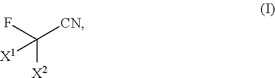

are reacted after preparation according to the method described above directly with sodium azide in the presence of a solvent to give the corresponding fluoroalkyltetrazoles of the general formula (III)

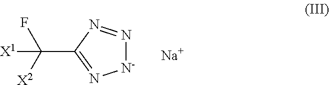

in which X¹ and X² are as defined above.

The amount of sodium azide for this step should be sufficiently large such that the nitrile can be completely converted in an industrially viable time. The molar ratio of sodium azide to fluoroalkylnitrile of the general formula (I) is preferably between 1 and 10, more preferably between 1 and 5 and particularly preferably between 1 and 2. The amount of solvent in which the azide salt is dissolved or suspended is not critical. Typical mixtures may comprise up to 20% by weight of azide.

Surprisingly, the fluorinated alkylnitriles of the formula (I) and the corresponding fluoroalkyltetrazoles of the general formula (III) prepared therefrom can be prepared safely and with good yields in high purity under the conditions according to the invention, which means that the method according to the invention does not have the disadvantages described in the context of the prior art.

### General Definitions

In the context of the present invention, the term halogens or halides includes, unless defined differently, elements selected from the group consisting of fluorine, chlorine, bromine and iodine, preference being given to using fluorine, chlorine and bromine, and particular preference to using fluorine and chlorine.

Optionally substituted groups may be mono- or polysubstituted, where the substituents in the case of polysubstitutions may be the same or different.

Phosphorus Trichloride (PCl₃) and/or Phosphorus Oxychloride (POCl₃)

To prepare fluoroalkylnitriles of the general formula (I), preference is given to using, inter alia, phosphorus trichloride (PCl₃) and/or (preferably or) phosphorus oxychloride (POCl₃).

The molar ratio of phosphorus trichloride (PCl₃) or phosphorus oxychloride (POCl₃) to the fluorinated alkylamide used of the general formula (II) may be, for example, from 0.05 to 1, preferably 0.5 to 0.9. The use of larger amounts (molar ratios greater than 1) of phosphorus trichloride (PCl₃) or phosphorus oxychloride (POCl₃) is not critical but uneconomical.

### Base

The method according to the invention for preparing fluoroalkylnitriles of the general formula (I) is carried out in the presence of a base. For example, pyridine or substituted pyridines and substituted or unsubstituted quinolines are suitable as bases. Preference is given to using pyridine or substituted pyridines and substituted or unsubstituted quinolines. Particularly preferred examples of bases are pyridine, picoline, quinoline, quinaldine and halogenated pyridines. Very particular preference is given to using 3-picoline.

The molar ratio of base to fluorinated alkylamide used of the general formula (II) may be, for example, from 1 to 10, particularly preferably 3 to 6.

The use of larger amounts of base is not critical but uneconomical.

The reaction for preparing compounds of the general formula (I) or also the subsequent reaction to compounds of the general formula (III) may generally be carried out under reduced pressure, at standard pressure or under elevated pressure. The temperatures applied may also be varied, depending on the substrates used, and can be readily determined by routine tests by those skilled in the art. For example, the reaction for the preparation of compounds of the general formula (I) may be carried out at a temperature from −50 to 250° C., preferably 0 to 170° C. The reaction is particularly preferably carried out at temperatures of 10 to 140° C.

The fluorinated alkylamides of the formula (II) used in accordance with the present invention are commercially obtainable or can be readily prepared by methods known from the literature (WO 03/080563).

### Solvent

The reaction of the fluorinated alkylamide of the formula (II) to give the compound of the formula (I) can optionally be carried out in the presence of a solvent. An additional solvent is preferably omitted in the reaction.

The reaction of the fluorinated alkylnitrile of the formula (I) to give the compound of the formula (III) is carried out in the presence of a solvent. For this reaction preference is given to using aprotic polar solvents such as, for example, a ketone such as acetone, a lactone such as γ-butyrolactone, a lactam such as N-methyl-2-pyrrolidone, a nitrile such as acetonitrile, a nitro compound such as nitromethane, a tertiary carboxamide such as dimethylformamide, a urea derivative such as tetramethylurea or a dimethylpropylene urea (DMPU), a sulphoxide such as dimethyl sulphoxide (DMSO), a sulphone such as sulpholane, a carbonic ester such as dimethyl carbonate or an ethylene carbonate. Particular preference is given to using acetone or acetonitrile as solvent.

The desired compounds of the general formula (I) may be isolated, for example, by distillation.

The desired compounds of the general formula (III) may be isolated, for example, by filtration.

The present invention is elucidated in detail by the examples which follow, although the examples should not be interpreted in such a manner that they restrict the invention.

### PREPARATION EXAMPLES

### Example 1

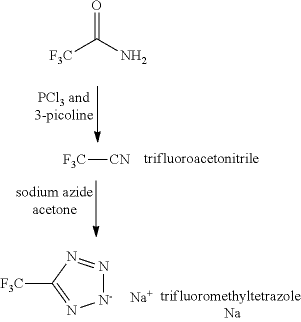 [SMILES: NC(=O)C(F)(F)F ; N#CC(F)(F)F ; FC(F)(F)c1nn[n-]n1 ; [Na+]]

A mixture of 148.3 g (1.592 mol, 3.00 eq.) of 3-picoline and 8.1 g (0.053 mol; 0.10 eq.) of POCl₃ was heated to 100° C. To this mixture were metered in 57.0 g (0.372 mol; 0.70 eq.) of POCl₃ and at the same time a mixture of 60.0 g (0.531 mol; 1.00 eq.) of trifluoroacetamide in 98.9 g (1.062 mol; 2.00 eq.) of 3-picoline over a period of 4 hours. Subsequently, the temperature was increased to 125° C. over a period of one hour. The resulting trifluoroacetonitrile is conducted through a reflux condenser and a wash bottle, filled with 3-picoline, and introduced into a mixture of 36.2 g (0.557 mol; 1.05 eq.) of sodium azide and 320.3 g of acetone. The slightly exothermic reaction is carried out at a temperature of 25° C. to 30° C. At the end of the reaction, the excess sodium azide is filtered off and washed with acetone. The filtrate is concentrated to 30% by weight of trifluoromethyltetrazole Na by distillation of acetone under reduced pressure. The theoretical yield is 75%.

### Example 2

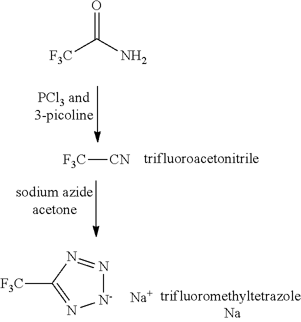 [SMILES: NC(=O)C(F)(F)F ; N#CC(F)(F)F ; FC(F)(F)c1nn[n-]n1 ; [Na+]]

A mixture of 150.0 g (1.613 mol; 3.00 eq.) of 3-picoline and 7.3 g (0.053 mol; 0.10 eq.) of PCl₃ was heated to 70° C. To this mixture were metered in 51.0 g (0.372 mol; 0.70 eq.) of PCl₃ and at the same time a mixture of 60.0 g (0.531 mol; 1.00 eq.) of trifluoroacetamide in 100.0 g (1.074 mol; 2.00 eq.) of 3-picoline over a period of 4 hours. Subsequently, the temperature was increased to 80° C. over a period of one hour. The resulting trifluoroacetonitrile is conducted through a reflux condenser and a wash bottle, filled with 3-picoline, and introduced into a mixture of 36.2 g (0.557 mol; 1.05 eq.) of sodium azide and 320.0 g of acetone. The slightly exothermic reaction is carried out at a temperature of 25° C. to 30° C. At the end of the reaction, the excess sodium azide is filtered off and washed with acetone. The filtrate is concentrated to 30% by weight of trifluoromethyltetrazole Na by distillation of acetone under reduced pressure. The theoretical yield is 63%.

1. Method for preparing fluoroalkylnitriles of formula (I), 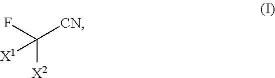
in which
X¹ and X² are mutually independently fluorine, chlorine, hydrogen or methyl,
comprising reacting
one or more fluorinated carboxamides of formula (II) 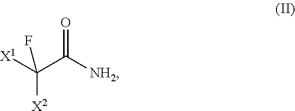
in which X¹ and X² are as defined above,
with phosphorus trichloride (PCl₃) and/or phosphorus oxychloride (POCl₃) in the presence of a base.

2. Method according to claim 1, wherein X¹ and X² are both fluorine.

3. Method according to claim 1, wherein the molar ratio of phosphorus halide to the fluorinated alkylamide used of formula (II) is 0.05 to 1.

4. Method according to claim 1, wherein the base is selected from the group consisting of pyridine, picoline, quinoline, quinaldine and halogenated pyridines.

5. Method according to claim 1, wherein the molar ratio of base to the fluorinated alkylamide used of formula (II) is 1 to 10.

6. Method according to claim 1, comprising reacting one or more fluoroalkylnitriles obtained of formula (I), 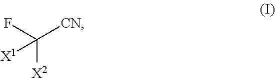
with sodium azide in the presence of a solvent to give the corresponding fluoroalkyltetrazoles of formula (III) 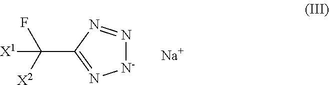
in which X¹ and X² are as defined above.
